(19) United States
(12) Patent Application Publication
(10) Pub. No.: US 2010/0111373 A1
(43) Pub. Date: May 6, 2010

(54) MEAN CURVATURE BASED DE-WEIGHTING FOR EMPHASIS OF CORNEAL ABNORMALITIES

(75) Inventors: Rodney P. CHIN, Carson City, NV (US); Rick A. WILLIAMS, Orinda, CA (US)

Correspondence Address: Carl Zeiss Meditec c/o MoFo, 425 Market Street, San Francisco, CA, 94105-2482, US

(73) Assignee: Carl Zeiss Meditec, Inc., Dublin, CA (US)

(21) Appl. No.: 12/566,916

(22) Filed: Sep. 25, 2009

Related U.S. Application Data
(60) Provisional application No. 61/111,899, filed on Nov. 6, 2008.

(51) Int. Cl. G06K 9/46; A61B 3/107; G06T 7/00

(52) U.S. Cl. 382/113; 351/246; 351/205; 382/203

(57) ABSTRACT

One embodiment of the present invention is a method for providing a deviation map of an eye that includes: (a) acquiring data comprising a corneal topographic map; (b) determining locations of abnormal curvature within the topographic map; (c) determining a modified topographic map by removing data associated with locations of abnormal curvature; (d) computing a reference map from the modified topographic map; (e) computing a deviation map by combining the topographic map and the reference map; and (f) displaying or storing the deviation map. An additional embodiment includes the processing of pachymetric data in a similar fashion.

### PRIORITY

This application claims priority to U.S. Provisional Application Ser. No. 61/111,899, filed Nov. 6, 2008, the disclosure of which is incorporated herein by reference.

### TECHNICAL FIELD

One or more embodiments of the present invention relate generally to visualization methods for corneal topographer data presentation, and in particular, a method for providing a means to display atypical corneal shape features separated from the typical (normal or basic) corneal map features.

### BACKGROUND

Topography is a process for mapping surfaces, such as a contour map of land. Similarly, corneal topography maps the corneal surface. The corneal curvature may be computed from corneal topography data that is obtained from a variety of existing measurement modalities. Placido-disk technology, Schleimpflug technology, or Optical Coherence Tomography (OCT) possess sufficient precision, accuracy, and resolution for this purpose. A healthy cornea consists of a clear layer acting as a window in the anterior portion of the eye and is responsible for about 70 percent of the eye's focusing power. This corneal window needs to be smooth and evenly rounded for good vision. An atypical cornea is a cornea that is too flat, too steep, bumpy, rippled, or otherwise unevenly shaped. Such corneas may cause defective vision or may represent inherent weaknesses that contraindicates refractive surgery, such as LASIK. Corneal topographer map displays produce detailed characterizations of the shape and refractive power of the cornea.

Optical Coherence Tomography (OCT) is an imaging modality used for non-invasive human eye imaging. An OCT imaging apparatus is capable of performing micrometer-resolution, cross-sectional imaging of the cornea. The OCT device uses a sample illumination light-source, an interferometer, and a light detector. The optical path length is determined by interferometry between the reflected light from the sample and the light from the reference light beam path. The precision, accuracy, and high-resolution of the OCT technique can produce highly-accurate corneal topography.

OCT technology was first developed in the early 1990's (see U.S. Pat. No. 5,321,501). This original technology has come to be known as time domain OCT (TD-OCT) in order to distinguish it from an alternative technology that has been more recently demonstrated, commonly called frequency domain OCT (FDOCT). FD-OCT has significant advantages in speed and signal to noise ratio as compared to TD-OCT (Leitgeb, R. A., et al., Optics Express 11:889-894). FD-OCT can be implemented either using a wideband source and a spectrometer (this variant is commonly called Spectral Domain OCT (SD-OCT) and also sometimes also referred to as Spectral Radar (J. Biomed. Optics, Vol. 3, No. 1 (1998) 1087-1089), or alternatively by sweeping a narrowband light source (this variant is commonly called Swept Source OCT (SS-OCT)). While there are advantages and disadvantages to each of these imaging techniques, they can each provide micron resolution imaging of the corneal surface.

Corneal measurements traditionally determine axial curvature (also called sagittal curvature) and tangential curvature (also called meridional curvature). These maps are confounded by the effects of normal corneal shapes affected by basic astigmatism and asphericity. Some techniques determine an average curvature, or the average curvature along a plurality of meridians (“directional curvature”), while other techniques compute local curvature at a plurality of points on the corneal surface. Generally, curvature is determined from the rate of change of the surface topography and can be related to a corresponding equivalent spherical radius. In particular, corneal topographers such as the Orbscan® corneal topographer and the ATLAS™ corneal topographer have been offering different “mean curvature” maps highlighting local regions of atypical steep curvature yet discount the underlying basic corneal shape. Unfortunately, “mean curvature” maps are not intuitive to the clinician and are not generally established in medical practice.

As is well known from the literature, surface derivatives describing the rate of change of the surface at a location are highly susceptible to variations in measurement values.

In light of the above, there is a need to develop clinically effective displays to highlight atypical local corneal variations within a traditional topographic format. The present invention offers methods and means to fulfill this goal.

### SUMMARY

The present invention is defined by the claims and nothing in this section should be taken as a limitation on those claims. Advantageously, embodiments of the present invention overcome the above-described problems in the art and provide methods of suppressing uninteresting topographic features or enhancing clinically interesting and relevant corneal features, or both.

### BRIEF DESCRIPTION OF THE DRAWINGS

FIGS. 1a to 1e illustrate the process of localization of an atypical region of steep curvature.

FIG. 2 illustrates the improved presentation in the residual map of an anterior irregularity. FIG. 2a illustrates the residual map computed from a reference surface derived using equally weighted data. FIG. 2b illustrates the residual map computed from a reference surface where atypical data is weighted differently from typical data.

FIG. 3 illustrates the improved presentation in the residual map of a posterior irregularity. FIG. 3a illustrates the residual map computed from a reference surface derived using equally weighted data. FIG. 3b illustrates the residual map computed from a reference surface where atypical data is weighted differently from typical data.

FIG. 4 illustrates the improved presentation in the pachymetry float map. FIG. 4a illustrates the residual map computed from a reference surface derived using equally weighted data. FIG. 4b illustrates the residual map computed from a reference surface where atypical data is weighted differently from typical data.

FIG. 5 illustrates the improved presentation in the elevation derivation from a sphere map. FIG. 5a illustrates the residual map computed from a spherical surface derived using equally weighted data. FIG. 5b illustrates the residual map computed from a spherical surface where atypical data is weighted differently from typical data.

FIG. 6 illustrates one design of an optical coherence tomography system.

### DETAILED DESCRIPTION

It should be understood that the embodiments, examples and descriptions have been chosen and described in order to illustrate the principles of the invention and its practical applications and not as a definition of the invention. Modifications and variations of the invention will be apparent to those skilled in the art. The claims define the scope of the invention, which includes known equivalents and unforeseeable equivalents at the time of filing of this application.

To first order, the corneal surface can be modeled as a portion of the surface of a sphere. A spherical surface has a constant curvature throughout. Hence, the local curvature of the typical cornea is a slowly varying, bounded function over its lateral extent. A simple threshold can be set to determine typical versus atypical local curvature values for a cornea. For example, the threshold can be set as a function of the difference of the local measured curvature of the cornea and the expected (typical) local curvature. If the threshold is set based on a constant curvature model, it should be set sufficiently robust so that it may also account for the normal variation in the curvature of the cornea.

While such a simple threshold is useful for many instances, it can be important to refine the technique to account for second order variations in the typical curvature of the cornea. This can be accomplished either by comparing corneal measurements to a general corneal model, or by simply looking at local variations of the corneal measurements. Examining local variations ignores variations in curvature at a distance. In other words, since the typical corneal curvature is a slowly varying function, rapid changes in measured curvature (bumps) are atypical. A measurement threshold can be set based on measurements of typical local variations of the mean curvature over a characteristic scale, such as a lateral length scale. Such thresholds may be set differently at different locations and detect atypical variations differently over different regions of the cornea.

Curvature is related to the radius of the circle that best approximates a one-dimensional cross section of the surface at a point. At a point on the surface, the curvature in each direction may be different. A good approximation of all of the curvatures to a physically unbroken surface at a point is the average of the various directional curvatures at that point. A map of the average of the directional curvatures at each point on the surface is called a mean curvature map. A mean curvature map may be computed from corneal elevation data or a topographic map. A mean curvature map may be used to identify and emphasize potential corneal abnormalities for presentation in conventional topographic (elevation) or pachymetric (thickness) corneal maps. Certainly, one method for computing a simple threshold over the entire cornea is to estimate the normal deviation from the average curvature of the mean curvature map.

For example, the threshold can be set as a function of the difference between the maximum mean curvature and the average mean curvature of the cornea, or

threshold=maximum curvature−k*(maximum curvature−mean curvature).

When k=1, the threshold is equal to the mean curvature. When k=0.5, the threshold is halfway between the maximum and the mean curvature. A second level threshold can also be included to inhibit the de-weighting when the ratio between the mean curvature and the maximum curvature is greater than a certain percentage (for example 97%), indicating a generally smooth, well-behaved corneal surface.

Alternatively, purely statistical methods can be used, where the threshold is set to between 1-3 standard deviations from the average mean curvature. Similarly, for regional variations, a threshold can be set at 1-3 standard deviations from the regional average mean curvature. Similarly, these methods can be applied using the corneal model instead of the empirically measured regional curvatures. Using the corneal model, the threshold is set to between 1-3 standard deviations from the predicted value based on the model.

Once a local curvature is determined to be typical or atypical, a simple binary mask can be set to include data in the region of location of typical curvature and exclude data in the region of location of atypical curvature from the computation of a reference topography map or a reference pachymetry map. The fit process to generate the reference surface utilizes the typical areas of the topographic or the pachymetric (residual/float) maps. Excluding regions of atypical curvature from the fitting provide maps that are unperturbed by any atypical corneal regions.

Alternatively, a de-weighting mask that is more complicated than a simple binary mask, can be created. Since normal corneal features are locally smooth within regions, a non-binary de-weighting can be generated as a function of the measured curvature and the expected curvature. Typical (normal) regions are weighted more heavily than those regions that are atypical. The more atypical the region, the more severely the weighting is devalued with regions identified as outliers having their weight most severely devalued. The binary mask is the simplest de-weighting mask with typical areas weighted 1 (unity) and atypical or outlier regions weighted 0 (null). The weights are applied to the constraints of the fit and not the data. For example, when using the simple binary mask during curve or surface fitting procedures, the null weighted regions are not included as zeros, but rather are excluded completely from the fit. When using a non-binary weighting, a certain percentage of data points within the mask region may be removed from the fit, or a higher density of data points outside of the mask may be included in the fit. In general, the de-weighting mask is applied to weight the influence of fit at the location to the goodness of fit measure.

Topographic or pachymetry maps generate the reference surfaces using de-weighted data. Float maps generated using a fitting process that minimizes or excludes atypical data from the fitting generates maps unperturbed by atypical corneal regions. Additionally, higher-order reference surfaces are able to be fit than could be done without the de-weighting mask. Without de-weighting, attempting to use higher-order reference surface fits tend to “absorb” the local atypical corneal features and reduce the amplitude of any potential “corneal abnormality”.

In some embodiments, a two pass algorithm is used to estimate the reference surface. A first pass estimate is performed using one set of data. Regions of atypical curvature are determined and a second pass estimate of the reference surface is computed. In the second pass, data points with atypical curvature are de-weighted or completely eliminated from the second pass computation of the reference surface.

Once the reference surface has been determined, the residual/float map is created from the original data by subtracting the fitted reference values. Because localized atypical corneal features are usually bumps or divots modifying the basic shape, by computing the reference surface without the use of the atypical data, minimal circumferential artifacts are generated in the residual map around the atypical regions. In conventional fitting, using the atypical data when generating the reference surface tends to create artifacts (such as moats or levees) in the region of the residual map near the atypical data region. Computing residual/float maps using de-weighted data results in a full amplitude visibility of a potential corneal abnormality with negligible artifacts. Mean curvature maps are not new. Products such as Bausch & Lomb's Orbscan® Anterior Segments Analysis System and Carl Zeiss' Humphrey® ATLAS™ Corneal Topography System have used mean curvature maps of one form or another for some years now. Some clinicians/doctors have adopted the use of these maps in their practice. However, these systems do not weight or de-weight data, based on the determination of regions of atypical data, before computing their residual maps.

Furthermore, the measure (units) of the degree of curvature is not physically intuitive and does not easily relate to a direct physical observable that is typically common in the ophthalmology practice. Hence, adoption of mean curvature maps is problematic.

More commonly, clinicians use (residual/float) elevation maps showing corneal topography differences from reference shapes such as spheres, ellipsoids, and aspheres. These conventional (residual/float) maps suffer from artifacts when using the model-dependent reference shapes. Also, there is ambiguity due to the arbitrary degree of “float” associated with the reference surface. The reference surface may float in the average position, in the minimum position, in the maximum position, or anywhere in between.

This new de-weighting technique benefits from the mean curvature operation on the typical topographic or pachymetric map and applies it to elevation or thickness residual/float maps, respectively. The resulting residual/float maps do not suffer from artifacts when using the model-dependent reference shapes. Since the atypical regions are removed from the reference surface fit, the reference surface is properly “floated” to the typical component of the corneal surface.

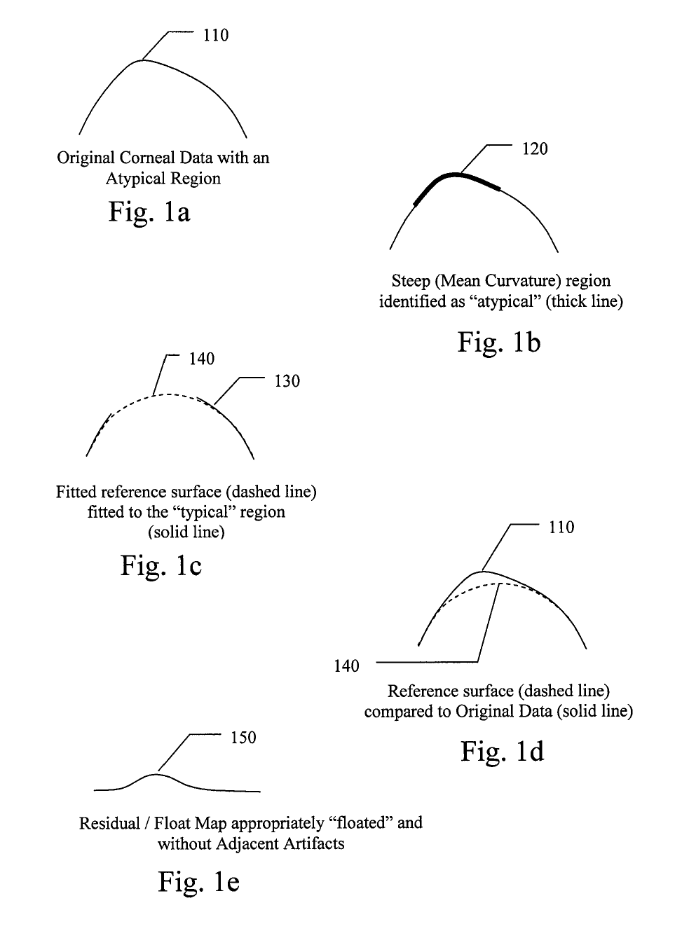

FIG. 1 graphically illustrates the basic process of de-weighting and reference surface fitting. FIG. 1a represents the corneal data 110. The thick line 120 in FIG. 1b bounds a region which has been identified as a region of atypical curvature. In accordance with the subject invention, this region of atypical curvature is de-weighted during the fitting procedure. In this particular example, the region 120 is masked, completely removing these points from consideration in the computation of the reference surface. The reference surface 140 is computed from the remaining points 130 of typical curvature. The computed reference surface and the original data shown in FIG. 1d are combined to create a residual map (sometimes also called a float map). The residual/float map shown in FIG. 1e is computed by taking the difference between the original corneal surface data 110 and the reference surface 140. The bump 150 in the cornea appears in the residual/float map without the adjacent artifacts (moats or levees) associated with reference/float maps created using reference maps generated using the atypical data regions.

The same technique applies to pachymetric data (thickness) maps. Localized regions of corneal thinning are detected as atypical. The atypical region is removed and the localized region is fully represented in a pachymetry (thickness) float map.

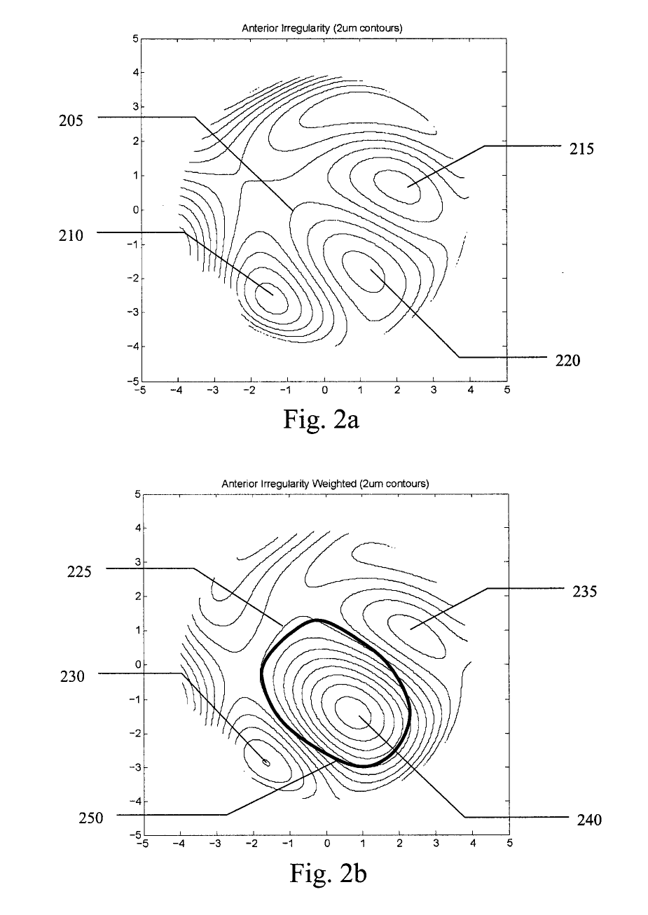

The patient data maps illustrated in FIGS. 2a and 2b demonstrate the advantage of the de-weighted fits on the anterior surface topographic maps. In FIG. 2a, the anterior irregularity is computed without weighting typical and atypical data differently in the derivation of the reference surface. The low points or troughs in the contour map 210 and 215 are separated from the peak 220 by a zero level contour 205. The peak 220 is less than 8 μm above the zero level contour. In FIG. 2b, the reference surface is computed by applying a mask to the atypical data. Data within the boundary 250 is determined to be atypical and the reference surface is computed using only data outside of the boundary 250. In the example depicted in this figure, data is determined to be atypical based solely on the mean curvature map. The zero level contour 225 is shifted (the zero level contour 205 of FIG. 2a more closely matches a 2 μm contour in FIG. 2b), the shape of the reference surface (not shown) has changed and now the peak 240 is greater than 16 μm above the zero level contour, even though the troughs 230 and 235 remain within a couple of microns (μms) of those shown in 2a.

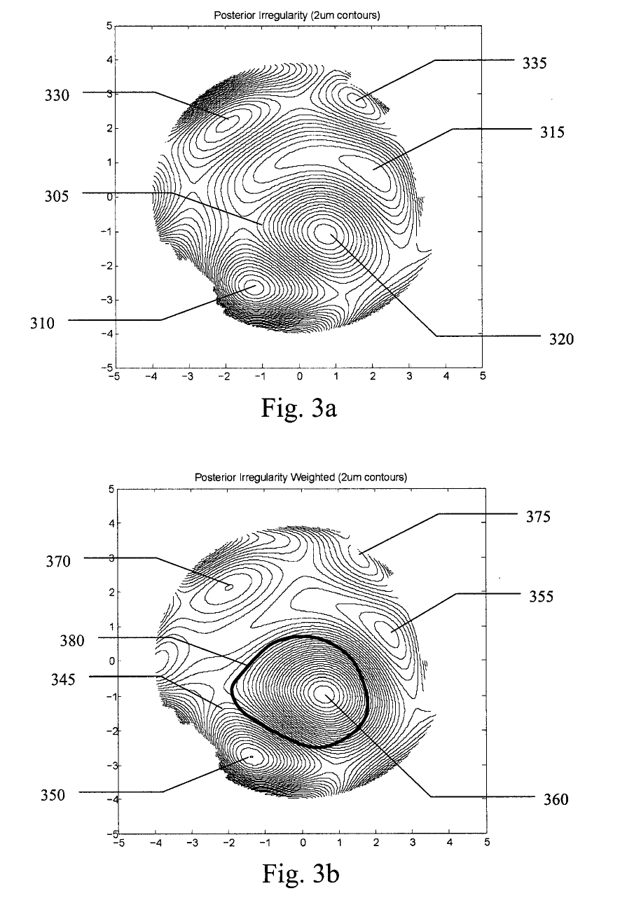

FIGS. 3a and 3b are similar to FIGS. 2a and 2b, except that FIG. 3 deals with a posterior irregularity while FIG. 2 dealt with an anterior irregularity. In FIG. 3a, the posterior irregularity is computed without weighting typical and atypical data differently in the derivation of the reference surface. The low points (moats or troughs) 310 and 315 in the contour map are separated from the peak 320 by a zero level contour 305. The peak 320 is less than 24 μm above the zero level contour. Artifact 315 is an anomalous trough more than 14 μm below the zero contour, while artifacts 330 and 335 are anomalous peaks, greater than 14 and 16 μm above the zero contour, respectively. In FIG. 3b, the reference surface is computed by applying a mask to the data to eliminate the atypical data. In the specific example depicted, data is determined to be atypical based on the mean curvature at the data location. Data within the boundary 380 is determined to be atypical and the reference surface is computed using only data outside of the boundary 380. The zero level contour 345 is significantly relocated from the zero contour 305. The peak 360 is greater than 50 μm above the zero level contour, approximately twice the height of the corresponding peak in FIG. 3a. Furthermore, the artifacts are significantly reduced. The though 355 (corresponding to 315) is only about 10 μm below the zero contour while peaks 370 and 375 (corresponding to 330 and 335, respectively) are approximately 8 and 12 μm high, respectively.

Maps created using reference surfaces derived from weighted data possess fewer artifacts than the maps created using reference surfaces derived from unweighted data and maps created using reference surfaces derived from weighted data provide a better estimate of the peak of the corneal anomaly. Artifacts caused by a mismatch of the reference surface when the reference surface is created using unweighted data are reduced or eliminated and the measurement of any inherent corneal anomaly is improved. In particular, when the data within the region of atypical curvature is removed from the process deriving the reference surface, the residual map shows greater peak variation and fewer artifacts than when the reference map is created using all of the data without weights to derive the reference surface.

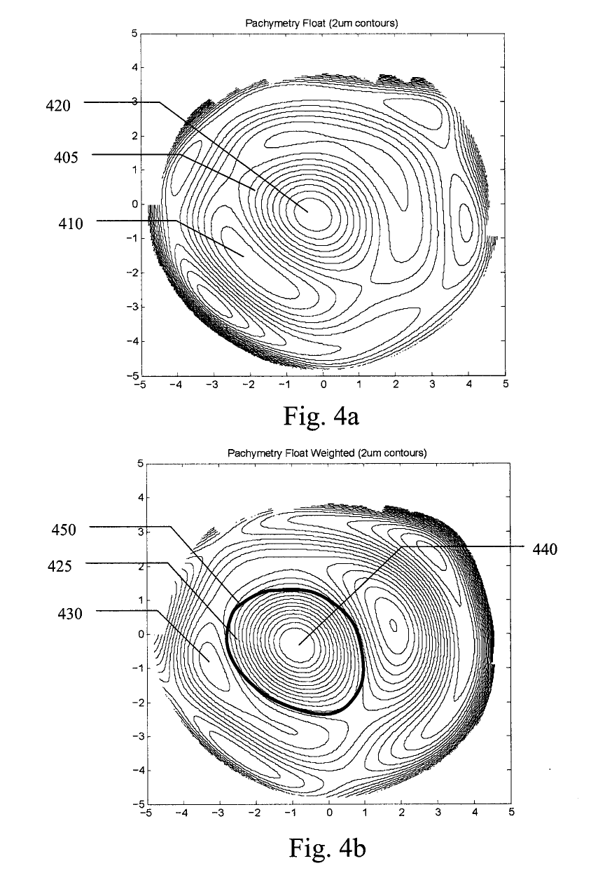

The pachymetry float maps illustrated in FIGS. 4a and 4b illustrate the advantage of applying de-weighted fits in computing the reference surface intermediate to deriving the pachymetry float maps. In FIG. 4a, the reference surface is computed without weighting typical and atypical data differently. The pachymetric corneal thinning 420 is clearly seen. Artifact 410 is an anomalous trough more than 8 μm below the zero contour 405. In FIG. 4b, the reference surface is again computed by applying a mask to the atypical data. Data within the boundary 450 is determined to be atypical and the reference surface is computed using only data outside of the boundary 450. The zero level contour 425 is significantly different in shape and location than the zero level contour 405. The peak 440 is greater than 20 μm and significantly higher than the corresponding peak 420 in FIG. 4a, which is less than 14 μm. The improved measure of the height of this peak is clinically significant.

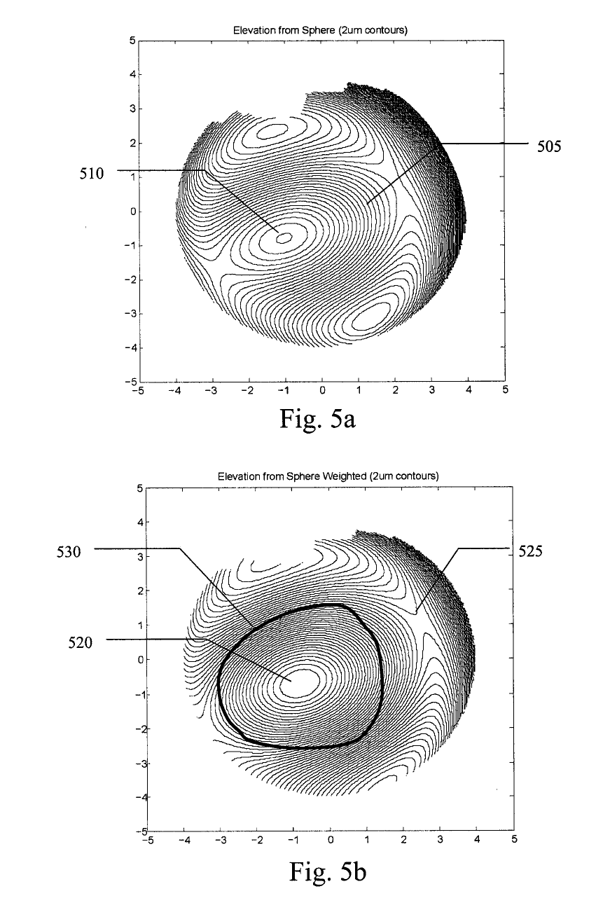

FIGS. 5a and 5b illustrate yet another embodiment of the present invention. Elevation data is often presented relative to a spherical surface fit to the cornea. In FIG. 5a, the spherical surface fit is performed using all of the data, while in FIG. 5b, the spherical surface fit takes advantage of applying a de-weighted fit. In FIG. 5a, the corneal elevation peak 510 is clearly visible and about 28 μm above the zero level contour 505. However, in FIG. 5b, after the spherical surface is computed without using atypical data, the peak 520 is over 60 μm above the zero level contour 525. Peak 520 is significantly elevated compared to the corresponding peak 510. For this example, data within the boundary 530 is determined to be atypical and the spherical surface is computed using only data outside of the boundary 530.

In general, better surface fits are made when atypical data is either not used or de-weighted when computing the reference surface. If the weighting for typical data is one, then the de-weighting coefficient for atypical data is in the interval [0, 1). (That is, in the interval between zero and one, including zero but not including 1.) A weighting of zero is equivalent to masking out the data and not using it at all. The usual care must be taken to ensure that the de-weighting coefficient de-weights the impact of the data on the resulting reference surface derivation and does not modify the value (usually here elevation) of that data.

Data can be determined to be atypical by using a model parameter, such as the local curvature. That is, if the local curvature at the data point differs from the model predicted curvature by more than a threshold, then the data point is atypical. Alternatively, data can be determined to be atypical on a purely statistical basis. These can be combined when the model contains a probability distribution so that the curvature can be identified as atypical if its value differs from the model-predicted value by, say, one standard deviation. In some applications, data may be identified as atypical if it is 2 or even 3 standard deviations from the expected mean.

Data can be determined to be atypical on the same pass through the data as is used to compute the reference surface or the determination of atypical data may be made on a separate pass through the data. In some instances, a preliminary reference surface may be computed from a portion or all of the data prior to determining which data points are typical and were are atypical. Of primary importance here is that, once a process is used to classify data points as typical or atypical, the reference surface is derived using data where atypical data is weighted differently than typical data. In extreme cases, all of the data may be classified as atypical. In these cases, it is critical that a weight is attached to measure the degree to which the data is atypical. If all of the data is atypical, then a simple mask eliminates all of the data so that no reference surface may be computed using the simple mask. Whenever much or all of the data is atypical, it is important that some atypical data be weighted by a weighting greater than zero.

The inventive method described herein is generally applicable to improving the fit of a smooth surface to any anatomical surface data, including, but not limited to, elevation data, thickness data, tissue layer data, boundary data, and/or image analysis data. Whenever a source of information is available for determining that individual data points are typical or atypical, whether such information is intrinsic to the data or based on external knowledge or heuristics, the classification of atypical data is useful in computing a reference surface fit to the data. Smooth reference surface fits are particularly useful. A reference surface fit to a retinal layer as described in U.S. patent application Ser. No. 11/223,549, filed Sep. 9, 2005 (publication No. 2007/0103693) which is incorporated herein by reference, can be improved by suitably de-weighting suspect data when performing the reference surface fit.

One method for setting the de-weighting co-efficient associated with a data location is by associating a probability of the goodness of the data with the data location. This can be implemented through the confidence maps described in U.S. patent application Ser. No. 11/978,184, filed Oct. 26, 2007 (publication No. 2008/0100612) which is incorporated herein by reference, or by local probability estimates associated with the variance of the mean curvature, or by other means known or recognizable to those versed in the art. Once the de-weighting coefficient is set, the de-weighting is applied to the influence of the data point on the computation of the reference surface. While, in some limited cases, this may be accomplished by modifying the value of the data point used in the derivation of the reference surface, in general it is not the data value that is de-weighted, but rather the impact that the data point has on the derivation of the reference surface that is de-weighted.

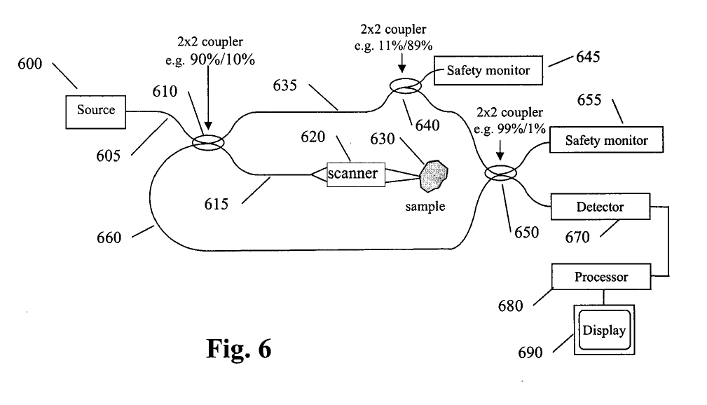

FIG. 6 illustrates an OCT device which can be used to implement the subject invention. Further information about this type of OCT device is disclosed in U.S. Patent Publication No. 2007/0291277, incorporated herein by reference. A low coherence light source 600, typically a superluminescent diode (SLD), is coupled to source fiber 605 that routes light to directional coupler 610. The optimal directional strength of the coupling depends on system design choices and may be 90/10 (as shown in FIG. 6) or 70/30 or other choices depending on SLD back reflection tolerance, the source illumination required to image the sample and other system design parameters. Directional coupler 610 splits the light into sample fiber 615 and reference fiber 635. The sample path may include a delay apparatus (not shown) to adjust the length of the sample path. The transverse scanner 620 deflects the OCT beam and preferably creates a focus in the beam near the region of interest in sample 630. The transverse scanner laterally scans the optical beam across the sample in order to image a volume of the sample.

Some light scattered from sample 630 returns through the scanner and delay apparatus to sample fiber 615. Coupler 610 routes this light through loop 660 to fiber coupler 650, where it interferes with the reference light. The combining coupler 650 provides two outputs. These outputs can be used for balanced detection (see U.S. Pat. No. 5,321,501 FIG. 10). Alternatively, the coupling ratio of coupler 650 can be adjusted to send most of the interfered light to a single OCT detector 670. Each OCT detector can be a single photodetector for use in time-domain OCT or swept-source OCT, or a spectrometer for use in spectral domain OCT.

Optional tap 640 diverts a fraction of the reference light to detector 645, which may be used to monitor the source power. Monitoring may be included to monitor the safety of the sample or to detect a degradation in the source 600. Additionally or alternatively, a monitoring detector 655 could be positioned at one output of the combining coupler 650. Alternatively, monitoring may not be included at all in the system. The tap removes some fraction of optical power from the reference fiber 635, reducing the power that reaches coupler 650. Sensitivity in OCT can reach the shot-noise limit if the reference power is large enough to bring the interference signal above receiver noise, but not so large as to bring intensity noise or beat noise above the level of shot noise.

The coupling ratios in directional couplers 610, 640 and 650 are chosen to set a safe level of illumination to the sample, and to set the appropriate reference power at the detector or detectors. For example, in the case of ophthalmic OCT of the retina using light with wavelengths near 850 nm, the safe exposure level is approximately 0.5 mW, and the optimum reference level at the detector is approximately 0.005 mW. Sources are available in this wavelength range having output power of approximately 5 mW. For these conditions one would use a coupling ratio near 90%/10% in the splitting coupler 610 so that 10% of the source power reaches the sample. 90% of the scattered light will then be routed to loop 660. In the case where there is a single OCT detector 670, the combining coupler 650 preferably routes most of the sample light to that detector. The splitting coupler routes 90% of source light, 4.5 mW, to reference fiber 635, while only 0.005 mW is required at the detector. One could use a combining coupler 650 that couples 0.1% of the reference light into the single OCT detector 670, but in manufacture it is difficult to control the 0.1% coupling factor. A preferred solution is to use a 99%/1% split ratio in combining coupler 650, and take advantage of the additional degree of freedom in tap 640 to adjust the reference power. Nominally, tapping 89% of the power form reference fiber 635 will provide an appropriate reference level of 0.005 mW at OCT detector 670, in this example.

As an alternative to adjusting the tap ratio of optional tap 640, one can adjust the reference level by including attenuating fiber (U.S. Pat. No. 5,633,974) in the reference path.

The output of the detector 670 is routed to processor 680. This processor may be a single device or a plurality of devices, preferentially optimized for their portion of processing. The processor 680 is connected to one or more peripherals providing a user interface devices, such as display 690. The processor might also be connected to other user interface devices (such as a keyboard, mouse, joystick, and others), and one or more external communication devices (such as a USB or network connector, optical storage media, printer, internet, and others), as well as possibly connecting to other imaging hardware (such as cameras, fixation targets, fundus viewers, and others) or peripheral patient devices (such as head support, height adjustment, and others) which are not shown. The processor 680 provides the computational power (in one or more modules) processing functions such as image formation, volume rendering, segmentation, registration, evaluation of cost functions, and/or other computational tasks required for medical imaging and analysis.

Data indicating the location of an anatomical region within the eye can be acquired using an OCT device such as the one described in US Patent Publication No. 2007/0291277 or in any topographer (such as the ATLAS™ corneal topographer) or other instrument providing elevation data, thickness data, location data, or any other data sufficient to describe the geometrical location of an anatomical region (e.g., cornea, retina), either in absolute coordinates or in relative coordinates. The subject method can be performed in real time with the on-board processor 680. Alternatively, it is possible to store the data for subsequent retrieval and processing either on the on-board processor 680 or on a separate processor such as a post processing workstation.

It should be understood that the embodiments, examples and descriptions have been chosen and described in order to illustrate the principles of the invention and its practical applications and not as a definition of the invention. Modifications and variations of the invention will be apparent to those skilled in the art. For example, alternative names may be used for atypical data such as: abnormal, unusual, uncharacteristic, peculiar, anomalous, bad, aberrant, inappropriate, irregular, etc. All equivalents of atypical are encompassed within the scope of the claims stipulated below. The scope of the invention is defined by the claims, which includes known equivalents and unforeseeable equivalents at the time of filing of this application.

The following references are hereby incorporated herein by reference.

### U.S. Patent Documents

U.S. Pat. No. 6,505,936 Holladay, et al. Ellipsoidal corneal modeling for estimation and reshaping

U.S. Pat. No. 6,033,396 Huang, et al. Apparatus and method for performing laser thermal keratoplasty with minimized regression

### U.S. Patent Publications

2006/0215114 Flachenecker. Local Average Curvature Map for Corneal Topographers

2007/0291277 Everett. Spectral domain optical coherence tomography system

2007/0103693 Everett. Method of bioimage data processing for revealing more meaningful anatomic features of diseased tissues

2008/0100612

### Other Publications

Leitgeb, R. A., et al. (2003). “Performance of Fourier domain vs. time domain optical coherence tomography.” Optics Express 11(8): 889-894.

Haeusler, G. and M. W. Lindner (1998). ““Coherence Radar” and “Spectral Radar”—New Tools for Dermatological Diagnosis.” Journal of Biomedical Optics 3(1): 21-31.

What is claimed is:

1. A method for determining corneal deviations from corneal elevation data acquired by an ophthalmic measurement device, said method comprising the steps of:
determining locations of atypical curvature within the elevation data;
computing a reference surface from the elevation data by de-weighting data associated with locations of atypical curvature;
computing a residual map as a function of the acquired corneal elevation data and the reference surface; and
displaying or storing the residual map.

2. A method as recited in claim 1, wherein during the step of computing the reference surface, the data associated with the locations of atypical curvature are fully de-weighted so that the data is excluded from the computation of the reference surface.

3. A method as recited in claim 1, wherein during the step of computing the reference surface, the data associated with the locations of atypical curvature are de-weighted as a function of the extent of the deviation from a typical curvature.

4. A method as recited in claim 1, wherein the step of computing the residual map includes a subtraction of the reference surface from the acquired corneal elevation data.

5. A method as recited in claim 1, wherein locations of atypical curvature are determined based on statistical deviation from the average curvature.

6. A method as recited in claim 1, wherein locations of atypical curvature are determined based on statistical deviation from the maximum curvature.

7. A method as recited in claim 1, wherein locations of atypical curvature are determined based on statistical deviation from a combination of the maximum curvature and the average curvature within a local corneal region.

8. A method as recited in claim 1, wherein locations of atypical curvature are determined based on a statistical deviation from the typical curvature shape for that cornea.

9. A method as recited in claim 1, wherein locations of atypical curvature are determined based on statistical deviation from the regional curvature based on a typical corneal model.

10. A method as recited in claim 1, wherein locations of atypical curvature are determined based on the lateral rate of change of the local curvature.

11. An apparatus for determining corneal deviations comprising:
a data acquisition device for acquiring corneal topographic data;
a processor for computing locations of atypical data within the topographic data and for determining a reference map from the topographic data by de-weighting the atypical data, said processor thereafter computing a residual map as a function of the reference map and the acquired corneal topographic data; and
a display for displaying the residual map.

12. An apparatus as recited in claim 11, wherein the processor excludes the atypical data when determining the reference map.

13. An apparatus as recited in claim 11, wherein when the processor determines the reference map, the atypical data associated are de-weighted as a function of the extent of the deviation from typical data.

14. An apparatus as recited in claim 11, wherein computation of the residual map is performed by subtracting the reference surface from the acquired corneal topographic data.

15. A method for determining corneal deviations from corneal topographic data acquired by an ophthalmic measurement device, said method comprising the steps of:
determining locations of atypical data within the topographic data;
computing a reference map from the topographic data by de-weighting the atypical data in the computation;
computing a residual map as a function of the reference map and the acquired topographic data; and
displaying or storing the residual map.

16. A method as recited in claim 15, wherein during the step of computing the reference map, the atypical data are fully de-weighted so that the data is excluded from the computation of the reference map.

17. A method as recited in claim 15, wherein during the step of computing the reference map, the atypical data are de-weighted as a function of the extent of the deviation from typical data.

18. A method as recited in claim 15, wherein the step of computing the residual map includes a subtraction of the reference map from the acquired topographic data.

19. A method for determining corneal deviations from corneal thickness data acquired by an ophthalmic measurement device, said method comprising the steps of:
determining locations of atypical thinning or thickening within the thickness data;
determining a modified pachymetry map from the thickness data by de-weighting data associated with locations of atypical thinning or thickening;
computing a residual map as a function of the modified pachymetry map and the corneal thickness data; and
displaying or storing the residual map.

20. A method as recited in claim 19, wherein during the step of determining the modified pachymetry map, the data associated with the locations of atypical thinning or thickening are fully de-weighted so that the data is excluded from the determination of the modified pachymetry map.

21. A method as recited in claim 19, wherein during the step of determining the modified pachymetry map, the data associated with the locations of atypical thinning or thickening are de-weighted as a function of the extent of the deviation from a typical thickness.

22. A method as recited in claim 19, wherein the step of computing the residual map includes a subtraction of the modified pachymetry map from the corneal thickness data.

23. A method of generating a map of a region within an eye, said eye being examined by a device capable of producing elevation data for an anatomical region within the eye, said method comprising the steps of:
acquiring data including location information of the anatomical region of the eye;
classifying data elements as typical or atypical;
deriving a reference surface from the data wherein the derivation assigns different weights based on whether the data is typical or atypical;
generating an image as a function of the reference surface and the acquired data; and
storing or displaying the image.

24. A method as recited in claim 23, wherein data elements are classified as typical or atypical based on a predetermined reference surface.

25. A method as recited in claim 23, wherein during the step of deriving a reference surface, the atypical data elements are fully de-weighted so that the atypical data elements are excluded from the derivation of the reference surface.

26. A method as recited in claim 23, wherein during the step of deriving the reference surface, the atypical data elements are de-weighted as a function of the extent of the deviation from typical data elements.

27. A method as recited in claim 23, wherein the step of generating an image includes a subtraction of the reference surface from the acquired data.
